(19) United States
(12) Patent Application Publication
(10) Pub. No.: US 2026/0314925 A1
(43) Pub. Date: Oct. 15, 2026

(54) METHOD AND SYSTEM FOR ACQUIRING TEAR SECRETION VOLUME

(71) Applicant: Eye & ENT Hospital of Fudan University, Shanghai (CN)

(72) Inventors: Jinhai HUANG, Shanghai (CN); Zhongxing CHEN, Shanghai (CN); Siheng LI, Shanghai (CN); Hang YU, Shanghai (CN); Xingtao ZHOU, Shanghai (CN); Xiaomin HUANG, Shanghai (CN)

(73) Assignee: Eye & ENT Hospital of Fudan University, Shanghai (CN)

(21) Appl. No.: 19/467,842

(22) Filed: Feb. 3, 2026

Related U.S. Application Data
(63) Continuation of application No. PCT/CN2025/108988, filed on Jul. 17, 2025.

(30) Foreign Application Priority Data
Nov. 6, 2024 (CN) 2024115754166

(51) Int. Cl. A61B 3/10; G06T 7/00

(52) U.S. Cl. CPC A61B 3/101; G06T 7/0012; G06T 2207/10012; G06T 2207/10024; G06T 2207/20172; G06T 2207/20221; G06T 2207/30041

(57) ABSTRACT

This disclosure relates to the technical field of liquid collection, and in particular, to a method and a system for acquiring tear secretion volume. The method includes: acquiring a tear meniscus image of a detected person, using a motion sensor and an image recognition algorithm to track the flow of a tear meniscus in real time, processing an acquired tear meniscus image, calculating a height value of the tear meniscus, performing analysis on received image data to determine whether the height value of the tear meniscus is normal, and allowing a user to view real-time data and a historical record of the height value of the tear meniscus through a touch screen, and perform system setting and parameter adjustment through a control button. According to this disclosure, the motion sensor and the advanced image recognition algorithm are combined.

### CROSS-REFERENCE TO RELATED APPLICATIONS

The application claims priority to Chinese patent application No. 2024115754166, filed on Nov. 6, 2024, the entire contents of which are incorporated herein by reference.

### TECHNICAL FIELD

This disclosure relates to the technical field of liquid collection, and in particular, to a method and a system for acquiring tear secretion volume.

### BACKGROUND

With the development of science and technology, people's attention to their own health is continuously increasing. Ocular health, as an important part of physical health, attracts more and more attention. Tear secretion volume is one of the important indicators reflecting ocular health, and its changes can indicate a variety of ocular diseases, such as xerophthalmia, keratitis, etc.

However, it has been found by the inventor that the technical solution still has at least the following defects:
1. In the measurement of the tear secretion volume, image acquisition usually relies on a single viewing angle or light source, which may lead to information loss or inconsistency in the acquired tear meniscus images under different illumination conditions. These methods may still fail to provide sufficient details and accuracy when dealing with complex illumination environments or dynamic changes.
2. Dynamic tracking in the prior art usually relies on a single algorithm to recognize and track the tear meniscus, which may not fully cope with complex tear meniscus flow and form changes. Traditional motion sensors and image recognition algorithms may have a problem of insufficient recognition precision, affecting the accuracy and real-time performance of a flow path of the tear meniscus.
3. In terms of image processing and analysis, image fusion in the prior art is usually limited to simple enhancement technologies, which may not fully highlight key features and details in the image.

### SUMMARY

Based on the above objectives, this disclosure provides a system for acquiring tear secretion volume.

A system for acquiring tear secretion volume, including an image acquisition module, a dynamic tracking module, an image processing module, a tear meniscus height calculation module, a data analysis module, and a user interaction module, where
the image acquisition module acquires a tear meniscus image of a detected person;
the dynamic tracking module uses a motion sensor and an image recognition algorithm to track the flow of a tear meniscus in real time, and updates the tear meniscus image in real time;
the image processing module processes an updated tear meniscus image;
the tear meniscus height calculation module calculates a height value of the tear meniscus based on a tear meniscus boundary extracted by the image processing module;
the data analysis module includes a data receiving unit, a data storage unit, and a result interpretation unit, where the data receiving unit is used for receiving image data transmitted by a data transmission module, the data storage unit is for used storing received image data and historical data, and the result interpretation unit performs analysis based on the received image data to determine whether the height value of the tear meniscus is normal; and
the user interaction module uses a touch screen and a control button to complete information interaction between a user and a system, and the user can view real-time data and a historical record of the height value of the tear meniscus through the touch screen, and perform system setting and parameter adjustment through the control button.

Optionally, the image acquisition module includes:
camera positioning: mounting a plurality of cameras at different angular positions of the tear meniscus respectively;
near-infrared light source arrangement: mounting a plurality of near-infrared light sources around the camera;
illumination condition adjustment: automatically adjusting brightness and a direction of the near-infrared light source according to a change in ambient light; and
synchronous shooting: coordinating each camera to simultaneously shoot the tear meniscus image within the same time through a synchronous control system.

Optionally, the dynamic tracking module includes:
motion sensor arrangement: arranging the motion sensor around the camera of the image acquisition module to capture a motion change in a tear meniscus area;
preliminary image acquisition: acquiring an initial tear meniscus image by using the image acquisition module to determine an initial position and a form of the tear meniscus;
image segmentation: segmenting an acquired tear meniscus image through the image recognition algorithm to extract a boundary and a main feature of the tear meniscus;
motion detection: capturing motion in the tear meniscus area by using the motion sensor, and generating motion data;
path recognition: combining the motion data provided by the motion sensor with the image recognition algorithm to recognize and track a flow path of the tear meniscus in real time;
image updating: tracking changes in the form and position of the tear meniscus by comparing consecutive image frames, and dynamically updating the tear meniscus image; and
error correction: correcting an error caused by noise or external interference during a tracking process.

Optionally, the image processing module includes:
image preprocessing: processing the updated tear meniscus image by using a denoising algorithm to eliminate random noise and background interference in the image, and reducing sharp edges and discontinuities in the image through an image smoothing technology;
feature extraction: adopting an edge detection algorithm to recognize and extract an edge feature in the tear meniscus image and determine a contour of the tear meniscus, and using a key point detection algorithm to recognize important feature points in the tear meniscus image, including upper and lower boundaries of the tear meniscus and a tear distribution;
image enhancement: using a contrast enhancement technology to increase brightness difference of different areas in the tear meniscus image, and adjusting a color of the tear meniscus image;
image correction: performing geometric correction on the tear meniscus image to eliminate an influence caused by camera angle and lens distortion, and adjusting brightness of different areas in the image by using a brightness equalization algorithm; and
image storage and transmission: compressing processed image data, and sending same to the data analysis module for further processing and analysis.

Optionally, the image enhancement includes:
image contrast enhancement: performing histogram equalization on the acquired tear meniscus image, and redistributing a gray value of the original image through an equalization algorithm;
equalization effect verification: performing visual verification and statistical analysis on an equalized image;
color adjustment: performing gamma correction on the equalized tear meniscus image, and adjusting the brightness of the image through a nonlinear transformation;
color adjustment effect verification: performing visual verification and brightness comparison analysis on a gamma-corrected image;
image fusion: performing fusion processing on an image subjected to contrast enhancement and color adjustment, and highlighting a key feature in the tear meniscus image through a further image processing technology; and
result storage and transmission: storing an enhanced tear meniscus image in a data storage unit, and transmitting enhanced image data to the data analysis module through the data transmission module for processing and analysis.

Optionally, the image fusion includes:
performing wavelet transform on the image: applying discrete wavelet transform to images I₁(x,y) and I₂(x,y) subjected to contrast enhancement and color adjustment respectively to obtain low-frequency and high-frequency coefficients of the image;
coefficient fusion, specifically including:
fusing the low-frequency coefficient: fusing basic structural information of the image by applying a weighted average or maximum value selection strategy to the low-frequency coefficient;
fusing the high-frequency coefficient: adopting a maximum value selection strategy for each type of high-frequency coefficient (LH,HL,HH) to retain detail and edge information of the image;
inverse discrete wavelet transform application: performing inverse discrete wavelet transform on a fused coefficient: performing inverse discrete wavelet transform on fused low-frequency and high-frequency coefficients to generate a fused image I_{fused}(x,y); and
image quality optimization: applying a further image enhancement technology to the fused image.

Optionally, the tear meniscus height calculation module includes:
boundary recognition: based on a tear meniscus image extracted by the image processing module, using Canny edge detection to recognize the upper and lower boundaries of the tear meniscus, and calculating a gradient magnitude;
boundary calibration: determining positions of upper and lower boundaries below the center of a pupil in the tear meniscus image, specifically by identifying the upper boundary where the tear meniscus contacts the cornea and the lower boundary where the tear meniscus contacts the eyelid margin, and marking these boundary points in the image to form upper and lower boundary lines of the tear meniscus;
boundary position extraction: extracting boundary position data from calibrated upper and lower boundary lines, including coordinates of the upper and lower boundaries;
height measurement: calculating a vertical distance between the upper and lower boundaries to obtain a height value of the tear meniscus; and
result correction: correcting a calculated height value to compensate for a deviation introduced due to an image acquisition angle or an image processing error.

Optionally, the data analysis module includes:
data receiving: a data receiving unit receiving tear meniscus image data from the data transmission module, the received data including an image file and related metadata thereof, and performing preliminary inspection on the received image data, followed by decoding and format conversion;
data storage: a data storage unit saving the received image data into a database of the system, including current image data and a processing result thereof, merging newly received image data with the historical data, and updating a data storage record;
result interpretation: extracting the height value of the tear meniscus from the stored data, and comparing same with a preset normal range;
classifying the interpretation result as “normal”, “mildly abnormal”, “moderately abnormal” or “severely abnormal”; and
report generation: generating an analysis report based on the interpretation result, including the height value of the tear meniscus, an interpretation conclusion, a trend analysis, and a suggestion.

Optionally, the user interaction module includes:
real-time data display: the touch screen displaying data of the height value of the tear meniscus in real time, including a current measurement value, a historical data chart, and statistical information, allowing a data display area on the touch screen to be dynamically updated according to the latest data transmitted by the data analysis module;
historical record viewing: the touch screen providing a function of accessing the historical record, allowing the user to choose to view a past data record and an analysis result, and using a screening tool on the touch screen to select a historical record within a specific time period or under a specific condition for viewing;
system setting: the touch screen providing a system setting interface, allowing the user to perform system configuration through the interface, including adjusting a parameter and selecting a display option, enabling that a modification performed by the user on the setting interface can be confirmed through the control button, and saving the setting into the system;
parameter adjustment: the control button allowing the user to adjust a system parameter, including a threshold of a normal range of a tear meniscus height and a data update frequency, and after the user adjusts the parameter, the system feeding back an adjustment effect in real time, and displaying corresponding confirmation information on the touch screen; and
operation feedback: the touch screen providing a confirmation prompt after the user completes an operation, and if an error occurs during the operation, the touch screen displaying error information and a solution suggestion.

A method for acquiring tear secretion volume, implemented by the system for acquiring tear secretion volume according to any one of claims 1 to 9, including the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: including a data receiving unit, a data storage unit, and a result interpretation unit, where the data receiving unit is used for receiving image data transmitted by a data transmission module, the data storage unit is used for storing received image data and historical data, and the result interpretation unit performs analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

Beneficial effects of this disclosure are as follows:

According to this disclosure, through the configuration of the plurality of cameras and near-infrared light sources, the tear meniscus image is acquired from different angles and illumination conditions, which greatly improves the integrity and consistency of the images. This multi-angle acquisition and illumination optimization technology ensures that a clear and comprehensive tear meniscus image can be acquired under various illumination conditions, providing a high-quality data foundation for subsequent image processing and analysis. This innovation significantly improves the precision and reliability of image acquisition, facilitating more accurate measurement of the tear secretion volume.

According to this disclosure, the motion sensor and the advanced image recognition algorithm are combined, so that the flow path of the tear meniscus can be recognized and tracked in real time. Through the comprehensive application, the dynamic tracking process is more accurate, and complex changes of the form and flow of the tear meniscus can be effectively handled. In addition, in this disclosure, through real-time image update and error correction, tracking precision and response speed of the system are improved, so that monitoring of tear meniscus flow is more real-time and accurate.

According to this disclosure, the image fusion is performed by using an optimized wavelet transform technology, and the low-frequency and high-frequency coefficients are finely processed, so that the overall quality and detail performance of the image are significantly improved. The wavelet transform fusion not only retains the basic structure of the image, but also enhances details and edges, ensuring the clarity and feature protrusion of the tear meniscus image. Furthermore, in this disclosure, combined with the further image enhancement technology, the visual effect of the image is optimized, providing high-quality data support for the accurate measurement and analysis of tear meniscus height.

### BRIEF DESCRIPTION OF THE DRAWINGS

To describe the technical solutions in this disclosure or in the prior art more clearly, the following briefly describes the accompanying drawings required for describing the embodiments or the prior art. Apparently, the accompanying drawings in the following description show merely some embodiments of this disclosure, and those of ordinary skill in the art may derive other accompanying drawings from these accompanying drawings without creative efforts.

FIG. 1 is a schematic diagram of a system for acquiring tear secretion volume according to an embodiment of this disclosure; and

FIG. 2 is a schematic diagram of a method for acquiring tear secretion volume according to an embodiment of this disclosure.

### DETAILED DESCRIPTION OF THE EMBODIMENTS

This disclosure is described in detail below with reference to the accompanying drawings and embodiments. Meanwhile, it should be noted here that, to make the embodiments more detailed, the following embodiments are preferred and optimal embodiments. For some known technologies, those skilled in the art may also adopt other alternative methods for implementation. Furthermore, the accompanying drawings are only intended to describe the embodiments more specifically, and are not intended to specifically limit this disclosure.

It should be pointed out that references in the specification to “one embodiment”, “an embodiment”, “an exemplary embodiment”, “some embodiments”, etc., indicate that the described embodiment may include a specific feature, structure, or characteristic, but not every embodiment necessarily includes the specific feature, structure, or characteristic. In addition, when the specific feature, structure, or characteristic is described in combination with an embodiment, implementing such a feature, structure, or characteristic in combination with other embodiments (whether explicitly described or not) shall be within the knowledge scope of those skilled in the relevant art.

Generally, terms may be understood at least partially from their use in the context. For example, depending at least in part on the context, the term “one or more” as used herein may be used to describe any feature, structure, or characteristic in a singular sense, or may be used to describe a combination of features, structures, or characteristics in a plural sense. In addition, the term “based on” may be understood as not necessarily intended to convey a set of exclusive factors, but instead, depending at least in part on the context, may allow for the existence of other factors not necessarily explicitly described.

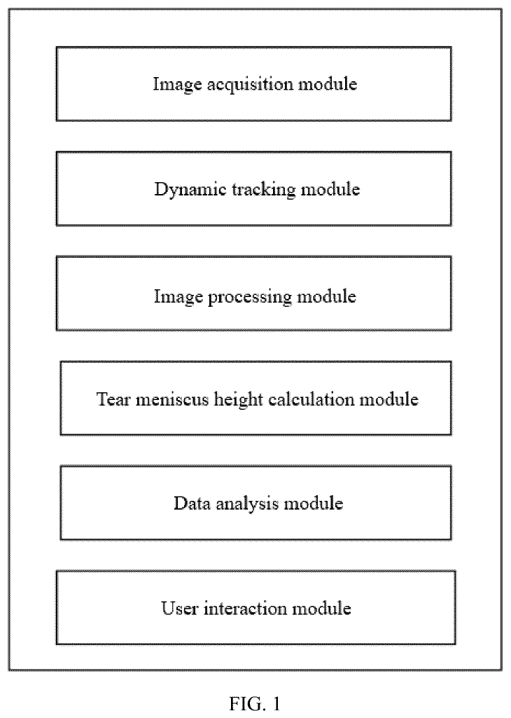

As shown in FIG. 1, a system for acquiring tear secretion volume includes an image acquisition module, a dynamic tracking module, an image processing module, a tear meniscus height calculation module, a data analysis module, and a user interaction module, where
the image acquisition module acquires a tear meniscus image of a detected person, including a camera and a near-infrared light source acquiring the tear meniscus image from different angles, and acquiring the tear meniscus image under different illumination conditions;
the dynamic tracking module uses a motion sensor and an image recognition algorithm to track the flow of a tear meniscus in real time, recognizes and tracks a flow path of the tear meniscus, and updates the tear meniscus image in real time;
the image processing module processes an updated tear meniscus image, and the image processing module includes an image preprocessing unit, a feature extraction unit, and an image enhancement unit, where the image preprocessing unit is used for removing noise and background interference, the feature extraction unit is used for extracting key features of the tear meniscus, and the image enhancement unit enhances the contrast and clarity of the image;
the tear meniscus height calculation module calculates a height value of the tear meniscus based on a tear meniscus boundary extracted by the image processing module, and the tear meniscus height calculation unit includes an automatic calibration unit and a height calculation module, where the automatic calibration unit is used for calibrating upper and lower boundaries of the tear meniscus below the center of a pupil, and the height calculation unit calculates a height value of the tear meniscus;
the data analysis module includes a data receiving unit, a data storage unit, and a result interpretation unit, where the data receiving unit is used for receiving image data transmitted by a data transmission module, the data storage unit is used for storing received image data and historical data, and the result interpretation unit performs analysis based on the received image data to determine whether the height value of the tear meniscus is normal; and
the user interaction module uses a touch screen and a control button to complete information interaction between a user and a system, and the user can view real-time data and a historical record of the height value of the tear meniscus through the touch screen, and perform system setting and parameter adjustment through the control button.

The image acquisition module includes:
camera positioning: mounting a plurality of cameras at different angular positions of the tear meniscus respectively to ensure that an entire area of the tear meniscus can be covered;
near-infrared light source arrangement: mounting a plurality of near-infrared light sources around the camera to ensure uniform illumination of the tear meniscus area under different illumination conditions, thereby reducing light interference;
illumination condition adjustment: automatically adjusting brightness and a direction of the near-infrared light source according to a change in ambient light, so as to optimize the illumination condition of the tear meniscus area; and
synchronous shooting: coordinating each camera to simultaneously shoot the tear meniscus image within the same time through a synchronous control system, ensuring the timeliness and consistency of the image.

Through the optimized arrangement of the multi-angle camera and the near-infrared light source, full coverage and uniform illumination of the tear meniscus area are ensured, thereby improving the clarity and accuracy of the image. These optimization measures improve the reliability and accuracy of image acquisition, providing high-quality data support for subsequent image processing and analysis.

The dynamic tracking module includes:
motion sensor arrangement: arranging the motion sensor around the camera of the image acquisition module to capture a motion change in a tear meniscus area;
preliminary image acquisition: acquiring an initial tear meniscus image by using the image acquisition module to determine an initial position and a form of the tear meniscus;
image segmentation: segmenting an acquired tear meniscus image through the image recognition algorithm to extract a boundary and a main feature of the tear meniscus;
motion detection: capturing motion in the tear meniscus area by using the motion sensor, and generating motion data;
path recognition: combining the motion data provided by the motion sensor with the image recognition algorithm to recognize and track a flow path of the tear meniscus in real time;
image updating: tracking changes in the form and position of the tear meniscus by comparing consecutive image frames, and dynamically updating the tear meniscus image; and
error correction: correcting an error caused by noise or external interference during a tracking process, ensuring the accuracy of the flow path of the tear meniscus.

The motion sensors are arranged around the cameras, so that accurate capture of motion changes within the tear meniscus area is achieved. Preliminary image acquisition and image segmentation technologies effectively extract an initial position and main features of the tear meniscus, laying a foundation for subsequent dynamic tracking. The motion detection and path recognition functions combine sensor data and the image recognition algorithm to achieve real-time tracking of the flow path of the tear meniscus. Image updating and error correction ensure the accuracy and reliability of the data.

The image processing module includes:
image preprocessing: processing the updated tear meniscus image by using a denoising algorithm to eliminate random noise and background interference in the image, and reducing sharp edges and discontinuities in the image through an image smoothing technology to improve image quality;
feature extraction: adopting an edge detection algorithm to recognize and extract an edge feature in the tear meniscus image and determine a contour of the tear meniscus, and using a key point detection algorithm to recognize important feature points in the tear meniscus image, including upper and lower boundaries of the tear meniscus and a tear distribution;
image enhancement: using a contrast enhancement technology to increase brightness difference of different areas in the tear meniscus image, making image details clearer, and adjusting a color of the tear meniscus image to enhance the visual effect of the image and highlight key features;
image correction: performing geometric correction on the tear meniscus image to eliminate an influence caused by camera angle and lens distortion, ensuring the accuracy of the image, and adjusting brightness of different areas in the image by using a brightness equalization algorithm to make the image more uniform and natural; and
image storage and transmission: compressing processed image data to reduce data volume and improve transmission efficiency, and sending same to the data analysis module for further processing and analysis.

Through multi-stage processing, the quality and analysis precision of the tear meniscus image are improved, the image quality is improved, key information in the image is enhanced, and the visual effect of the image is improved, so that the features are more prominent, the accuracy of the image is ensured, and the transmission efficiency of data is optimized.

The image enhancement includes:
image contrast enhancement: performing histogram equalization on the acquired tear meniscus image, and redistributing a gray value of the original image through an equalization algorithm, expressed as:

$$y = floor ( ( L - 1 ) \cdot \frac{{\sum }_{i = 0}^{x} p ( i )}{N} ) ;$$

where y is a gray value after equalization, L is the number of gray levels (usually 256), p(i) is the number of pixels with a gray value of i, N is the total number of pixels,

$${\sum }_{i = 0}^{x} p ( i )$$

is a cumulative distribution function of gray values from 0 to x;
equalization effect verification: performing visual verification and statistical analysis on an equalized image to ensure a significant contrast enhancement effect;
color adjustment: performing gamma correction on the equalized tear meniscus image, and adjusting the brightness of the image through a nonlinear transformation to make image details clearer, expressed as:

I′=I^{γ};
where I′ is a pixel value after gamma correction, I is a pixel value of the original image, a value range is usually between 0 and 1, γ is a gamma value, and a commonly used value is between 0.5 and 2.5;
color adjustment effect verification: performing visual verification and brightness comparison analysis on a gamma-corrected image to ensure that the visual effect of the color-adjusted image is significantly improved;
image fusion: performing fusion processing on an image subjected to contrast enhancement and color adjustment to ensure the details and visual effect of the image reach an optimal state, and highlighting a key feature in the tear meniscus image through a further image processing technology to improve the overall quality of the image; and
result storage and transmission: storing an enhanced tear meniscus image in a data storage unit to ensure the integrity and traceability of the image data, and transmitting enhanced image data to the data analysis module through the data transmission module for processing and analysis.

Through technologies such as contrast enhancement, color adjustment, and image fusion, the visual effect and analysis precision of the tear meniscus image are significantly improved, so that the details are clearer, and the gamma correction further improves the brightness and color performance of the image. The image fusion technology ensures the consistency and the optimal state of an enhanced image in terms of details and visual effect.

The image fusion includes:
performing wavelet transform on the image: applying discrete wavelet transform (DWT) to images I₁(x,y) and I₂(x,y) subjected to contrast enhancement and color adjustment respectively to obtain low-frequency and high-frequency coefficients of the image, expressed as;

DWT(Iᵢ(x,y))={LLᵢ,LHᵢ,HLᵢ,HHᵢ};
where LL is a low-frequency coefficient, LH, HL, and HH are respectively horizontal, vertical, and diagonal high-frequency coefficients;
coefficient fusion, specifically including:
fusing the low-frequency coefficient: fusing basic structural information of the image by applying a weighted average or maximum value selection strategy to the low-frequency coefficient, expressed as;

LL_{fused}=α·LL₁+(1−α)·LL₂
where a is a weighting coefficient;
fusing the high-frequency coefficient: adopting a maximum value selection strategy for each type of high-frequency coefficient (LH,HL,HH) to retain detail and edge information of the image, expressed as;

Coeff_{fused}=max(Coeff₁,Coeff₂);
where Coeff represents different types of high-frequency coefficients;
inverse discrete wavelet transform application: performing inverse discrete wavelet transform on a fused coefficient: performing inverse discrete wavelet transform (IDWT) on fused low-frequency and high-frequency coefficients to generate a fused image I_{fused}(x,y), expressed as

I_{fused}(x,y)=IDWT(LL_{fused},LH_{fused},HL_{fused},HH_{fused});
where IDWT is an inverse discrete wavelet transform operation;
image quality optimization: applying a further image enhancement technology (such as local contrast enhancement or noise suppression) to the fused image to further optimize image quality and highlight features in the tear meniscus image, expressed as:

I_{final}(x,y)=Enhance(I_{fused}(x,y));
where Enhance represents the image quality optimization technology; and
through the comprehensive processing of discrete wavelet transform, coefficient fusion, and inverse discrete wavelet transform, the overall quality and detail performance of the image are significantly improved. The discrete wavelet transform decomposes the image into low-frequency and high-frequency coefficients, and a basic structure and detail information of the image are respectively processed, so that the details and edges are clearer. The fusion of the low-frequency coefficients retains the main structure of the image, and the fusion of high-frequency coefficients enhances the details and edges of the image. The inverse discrete wavelet transform resynthesizes these fused coefficients into a final image, and optimizes the visual effect through the further image enhancement technology, thereby ensuring the clarity and feature protrusion of the tear meniscus image and providing high-quality data for accurate analysis and measurement.

The tear meniscus height calculation module includes:
boundary recognition: based on a tear meniscus image extracted by the image processing module, using Canny edge detection to recognize the upper and lower boundaries of the tear meniscus, and calculating a gradient magnitude, expressed as:

$$GradientMagnitude = \sqrt{{( \frac{\partial I}{\partial x} )}^{2} + {( \frac{\partial I}{\partial y} )}^{2}} ;$$

where

$$\frac{\partial I}{\partial x} and \frac{\partial I}{\partial y}$$

are gradients of the image in x and y directions respectively;
boundary calibration: determining positions of upper and lower boundaries below the center of a pupil in the tear meniscus image, specifically by identifying the upper boundary where the tear meniscus contacts the cornea and the lower boundary where the tear meniscus contacts the eyelid margin, and marking these boundary points in the image to form upper and lower boundary lines of the tear meniscus;
boundary position extraction: extracting boundary position data from calibrated upper and lower boundary lines, including coordinates of the upper and lower boundaries;
height measurement: calculating a vertical distance between the upper and lower boundaries to obtain a height value of the tear meniscus; and
result correction: correcting a calculated height value to compensate for a deviation introduced due to an image acquisition angle or an image processing error, ensuring the accuracy of the height value.

Through the comprehensive processing of boundary recognition, boundary calibration, boundary position extraction, height measurement, and result correction, the calculation process of the tear meniscus height can accurately measure the height value of the tear meniscus, thereby ensuring the accurate measurement of the tear meniscus height, and providing reliable data support for the evaluation of tear secretion volume.

The data analysis module includes:
data receiving: a data receiving unit receiving tear meniscus image data from the data transmission module, the received data including an image file and related metadata thereof, and performing preliminary inspection on the received image data to ensure data integrity and correctness, followed by decoding and format conversion;
data storage: a data storage unit saving the received image data into a database of the system, including current image data and a processing result thereof, merging newly received image data with the historical data, and updating a data storage record to facilitate trend analysis and comparison;
result interpretation: extracting the height value of the tear meniscus from the stored data, and comparing same with a preset normal range, expressed as;

$$interpretation result = \{ \begin{matrix}Normal & if {H}_{min} \leq H \leq {H}_{max} \\ Abnormal & if H < {H}_{min} or H > {H}_{max}\end{matrix} ;$$

where His a currently calculated value of the height value of the tear meniscus, and Hₘᵢₙ and Hₘₐₓ are lower and upper limits of the normal range;
classifying the interpretation result as “normal”, “mildly abnormal”, “moderately abnormal” or “severely abnormal”; and
report generation: generating an analysis report based on the interpretation result, including the height value of the tear meniscus, an interpretation conclusion, a trend analysis, and a suggestion, for subsequent medical decisions.

The user interaction module includes:
real-time data display: the touch screen displaying data of the height value of the tear meniscus in real time, including a current measurement value, a historical data chart, and statistical information, allowing a data display area on the touch screen to be dynamically updated according to the latest data transmitted by the data analysis module, ensuring that the user obtains the latest tear meniscus height information;
historical record viewing: the touch screen providing a function of accessing the historical record, allowing the user to choose to view a past data record and an analysis result, and using a screening tool on the touch screen to select a historical record within a specific time period or under a specific condition for viewing;
system setting: the touch screen providing a system setting interface, allowing the user to perform system configuration through the interface, including adjusting a parameter and selecting a display option, enabling that a modification performed by the user on the setting interface can be confirmed through the control button, and saving the setting into the system;
parameter adjustment: the control button allowing the user to adjust a system parameter, including a threshold of a normal range of a tear meniscus height and a data update frequency, and after the user adjusts the parameter, the system feeding back an adjustment effect in real time, and displaying corresponding confirmation information on the touch screen; and
operation feedback: the touch screen providing a confirmation prompt after the user completes an operation to ensure the accuracy of the operation, and if an error occurs during the operation, the touch screen displaying error information and a solution suggestion to help the user correct the operation.

Comprehensive information display and system setting functions are provided through the touch screen and the control button, which greatly improves user experience and operational convenience, ensuring that the user can use the system efficiently and accurately, and make effective adjustments and decisions.

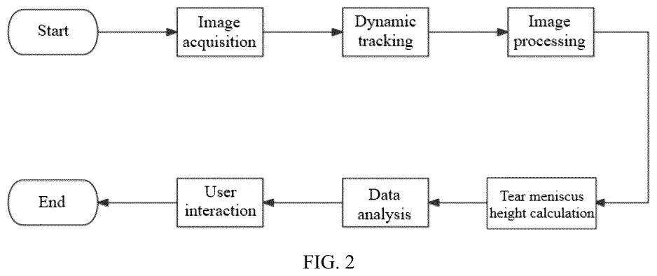

As shown in FIG. 2, a method for acquiring tear secretion volume, implemented by the above system for acquiring tear secretion volume, including the following steps:
S1, image acquisition: acquiring a tear meniscus image of a detected person, including a camera and a near-infrared light source acquiring the tear meniscus image from different angles, and acquiring the tear meniscus image under different illumination conditions;
S2, dynamic tracking: using a motion sensor and an image recognition algorithm to track the flow of a tear meniscus in real time, recognizing and tracking a flow path of the tear meniscus, and updating the tear meniscus image in real time;
S3, image processing: processing an updated tear meniscus image, and an image processing module including an image preprocessing unit, a feature extraction unit, and an image enhancement unit, where the image preprocessing unit is used for removing noise and background interference, the feature extraction unit is used for extracting key features of the tear meniscus, and the image enhancement unit enhances the contrast and clarity of the image;
S4, tear meniscus height calculation: calculating a height value of the tear meniscus based on a tear meniscus boundary extracted by the image processing module, and a tear meniscus height calculation module including an automatic calibration unit and a height calculation unit, where the automatic calibration unit is used for calibrating upper and lower boundaries of the tear meniscus below the center of a pupil, and the height calculation unit calculates a height value of the tear meniscus;
S5, data analysis: including a data receiving unit, a data storage unit, and a result interpretation unit, where the data receiving unit is used for receiving image data transmitted by a data transmission module, the data storage unit is used for storing received image data and historical data, and the result interpretation unit performs analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

This disclosure encompasses any substitutions, modifications, equivalent methods, and solutions made within the spirit and scope of this disclosure. To enable the public to have a thorough understanding of this disclosure, specific details are described in detail in the following preferred embodiments of this disclosure, and for those skilled in the art, without the description of these detailed parts, this disclosure can be fully understood. In addition, to avoid unnecessary confusion about the essence of this disclosure, well-known methods, processes, procedures, elements, circuits, etc. are not described in detail.

The above are merely preferred embodiments of this disclosure. It should be noted that, for those of ordinary skill in the art, a plurality of improvements and modifications can be made without departing from the principle of this disclosure, and these improvements and modifications are also regarded to be within the scope of protection of this disclosure.

What is claimed is:

1. A system for acquiring tear secretion volume, comprising an image acquisition module, a dynamic tracking module, an image processing module, a tear meniscus height calculation module, a data analysis module, and a user interaction module, wherein
the image acquisition module acquires a tear meniscus image of a detected person;
the dynamic tracking module uses a motion sensor and an image recognition algorithm to track the flow of a tear meniscus in real time, and updates the tear meniscus image in real time;
the image processing module processes an updated tear meniscus image;
the tear meniscus height calculation module calculates a height value of the tear meniscus based on a tear meniscus boundary extracted by the image processing module;
the data analysis module receives image data transmitted by a data transmission module, stores received image data and historical data, and performs analysis based on the received image data to determine whether the height value of the tear meniscus is normal; and
the user interaction module uses a touch screen and a control button to complete information interaction between a user and a system, and the user can view real-time data and a historical record of the height value of the tear meniscus through the touch screen, and perform system setting and parameter adjustment through the control button.

2. The system for acquiring tear secretion volume according to claim 1, wherein the image acquisition module comprises:
camera positioning: mounting a plurality of cameras at different angular positions of the tear meniscus respectively;
near-infrared light source arrangement: mounting a plurality of near-infrared light sources around the camera;
illumination condition adjustment: automatically adjusting brightness and a direction of the near-infrared light source according to a change in ambient light; and
synchronous shooting: coordinating each camera to simultaneously shoot the tear meniscus image within the same time through a synchronous control system.

3. The system for acquiring tear secretion volume according to claim 1, wherein the dynamic tracking module comprises:
motion sensor arrangement: arranging the motion sensor around the camera of the image acquisition module to capture a motion change in a tear meniscus area;
preliminary image acquisition: acquiring an initial tear meniscus image by using the image acquisition module to determine an initial position and a form of the tear meniscus;
image segmentation: segmenting an acquired tear meniscus image through the image recognition algorithm to extract a boundary and a main feature of the tear meniscus;
motion detection: capturing motion in the tear meniscus area by using the motion sensor, and generating motion data;
path recognition: combining the motion data provided by the motion sensor with the image recognition algorithm to recognize and track a flow path of the tear meniscus in real time;
image updating: tracking changes in the form and position of the tear meniscus by comparing consecutive image frames, and dynamically updating the tear meniscus image; and
error correction: correcting an error caused by noise or external interference during a tracking process.

4. The system for acquiring tear secretion volume according to claim 1, wherein the image processing module comprises:
image preprocessing: processing the updated tear meniscus image by using a denoising algorithm to eliminate random noise and background interference in the image, and reducing sharp edges and discontinuities in the image through an image smoothing technology;
feature extraction: adopting an edge detection algorithm to recognize and extract an edge feature in the tear meniscus image and determine a contour of the tear meniscus, and using a key point detection algorithm to recognize important feature points in the tear meniscus image, comprising upper and lower boundaries of the tear meniscus and a tear distribution;
image enhancement: using a contrast enhancement technology to increase brightness difference of different areas in the tear meniscus image, and adjusting a color of the tear meniscus image;
image correction: performing geometric correction on the tear meniscus image to eliminate an influence caused by camera angle and lens distortion, and adjusting brightness of different areas in the image by using a brightness equalization algorithm; and
image storage and transmission: compressing processed image data, and sending same to the data analysis module for further processing and analysis.

5. The system for acquiring tear secretion volume according to claim 4, wherein the image enhancement comprises:
image contrast enhancement: performing histogram equalization on the acquired tear meniscus image, and redistributing a gray value of the original image through an equalization algorithm;
equalization effect verification: performing visual verification and statistical analysis on an equalized image;
color adjustment: performing gamma correction on the equalized tear meniscus image, and adjusting the brightness of the image through a nonlinear transformation;
color adjustment effect verification: performing visual verification and brightness comparison analysis on a gamma-corrected image;
image fusion: performing fusion processing on an image subjected to contrast enhancement and color adjustment, and highlighting a key feature in the tear meniscus image through a further image processing technology; and
result storage and transmission: storing an enhanced tear meniscus image in a data storage unit, and transmitting enhanced image data to the data analysis module through the data transmission module for processing and analysis.

6. The system for acquiring tear secretion volume according to claim 5, wherein the image fusion comprises:
performing wavelet transform on the image: applying discrete wavelet transform to images I₁(x,y) and I₂(x,y) subjected to contrast enhancement and color adjustment respectively to obtain low-frequency and high-frequency coefficients of the image;
coefficient fusion, specifically comprising:
fusing the low-frequency coefficient: fusing basic structural information of the image by applying a weighted average or maximum value selection strategy to the low-frequency coefficient;
fusing the high-frequency coefficient: adopting a maximum value selection strategy for each type of high-frequency coefficient (LH,HL,HH) to retain detail and edge information of the image;
inverse discrete wavelet transform application: performing inverse discrete wavelet transform on a fused coefficient: performing inverse discrete wavelet transform on fused low-frequency and high-frequency coefficients to generate a fused image I_{fused}(x,y); and
image quality optimization: applying a further image enhancement technology to the fused image.

7. The system for acquiring tear secretion volume according to claim 1, wherein the tear meniscus height calculation module comprises:
boundary recognition: based on a tear meniscus image extracted by the image processing module, using Canny edge detection to recognize the upper and lower boundaries of the tear meniscus, and calculating a gradient magnitude;
boundary calibration: determining positions of upper and lower boundaries below the center of a pupil in the tear meniscus image, specifically by identifying the upper boundary where the tear meniscus contacts the cornea and the lower boundary where the tear meniscus contacts the eyelid margin, and marking these boundary points in the image to form upper and lower boundary lines of the tear meniscus;
boundary position extraction: extracting boundary position data from calibrated upper and lower boundary lines, comprising coordinates of the upper and lower boundaries;
height measurement: calculating a vertical distance between the upper and lower boundaries to obtain a height value of the tear meniscus; and
result correction: correcting a calculated height value to compensate for a deviation introduced due to an image acquisition angle or an image processing error.

8. The system for acquiring tear secretion volume according to claim 1, wherein the data analysis module comprises:
data receiving: a data receiving unit receiving tear meniscus image data from the data transmission module, the received data comprising an image file and related metadata thereof, and performing preliminary inspection on the received image data, followed by decoding and format conversion;
data storage: a data storage unit saving the received image data into a database of the system, comprising current image data and a processing result thereof, merging newly received image data with the historical data, and updating a data storage record;
result interpretation: extracting the height value of the tear meniscus from the stored data, and comparing same with a preset normal range;
classifying the interpretation result as “normal”, “mildly abnormal”, “moderately abnormal” or “severely abnormal”; and
report generation: generating an analysis report based on the interpretation result, including the height value of the tear meniscus, an interpretation conclusion, a trend analysis, and a suggestion.

9. The system for acquiring tear secretion volume according to claim 1, wherein the user interaction module comprises:
real-time data display: the touch screen displaying data of the height value of the tear meniscus in real time, comprising a current measurement value, a historical data chart, and statistical information, allowing a data display area on the touch screen to be dynamically updated according to the latest data transmitted by the data analysis module;
historical record viewing: the touch screen providing a function of accessing the historical record, allowing the user to choose to view a past data record and an analysis result, and using a screening tool on the touch screen to select a historical record within a specific time period or under a specific condition for viewing;
system setting: the touch screen providing a system setting interface, allowing the user to perform system configuration through the interface, comprising adjusting a parameter and selecting a display option, enabling that a modification performed by the user on the setting interface can be confirmed through the control button, and saving the setting into the system;
parameter adjustment: the control button allowing the user to adjust a system parameter, comprising a threshold of a normal range of a tear meniscus height and a data update frequency, and after the user adjusts the parameter, the system feeding back an adjustment effect in real time, and displaying corresponding confirmation information on the touch screen; and
operation feedback: the touch screen providing a confirmation prompt after the user completes an operation, and if an error occurs during the operation, the touch screen displaying error information and a solution suggestion.

10. A method for acquiring tear secretion volume, implemented by the system for acquiring tear secretion volume according to claim 1, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

11. A method for acquiring tear secretion volume, implemented by the system for acquiring tear secretion volume according to claim 2, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

12. A method for acquiring tear secretion volume, implemented by the system for acquiring tear secretion volume according to claim 3, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

13. A method for acquiring tear secretion volume, implemented by the system for acquiring tear secretion volume according to claim 4, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

14. A method for acquiring tear secretion volume, implemented by the system for acquiring tear secretion volume according to claim 5, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

15. A method for acquiring tear secretion volume, implemented by the system for acquiring tear secretion volume according to claim 6, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

16. A method for acquiring tear secretion volume, implemented by the system for acquiring tear secretion volume according to claim 7, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

17. A method for acquiring tear secretion volume, implemented by the system for acquiring tear secretion volume according to claim 8, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.

18. A method for acquiring tear secretion volume, implemented by the system for acquiring tear secretion volume according to claim 9, comprising the following steps:
S1, image acquisition: acquiring a tear meniscus image of a detected person;
S2, dynamic tracking: using a motion sensor and an image recognition algorithm to track the flow of a tear meniscus in real time, and updating the tear meniscus image in real time;
S3, image processing: processing an updated tear meniscus image;
S4, tear meniscus height calculation: calculating a height value of the tear meniscus based on a tear meniscus boundary extracted by the image processing module;
S5, data analysis: receiving image data transmitted by a data transmission module, storing received image data and historical data, and performing analysis based on the received image data to determine whether the height value of the tear meniscus is normal; and
S6, user interaction: using a touch screen and a control button to complete information interaction between a user and a system, and allowing the user to view real-time data and a historical record of the height value of the tear meniscus through the touch screen, and perform system setting and parameter adjustment through the control button.
